(19) United States
(12) Patent Application Publication
(10) Pub. No.: US 2019/0298502 A1
(43) Pub. Date: Oct. 3, 2019

(54) Measurement Device and a Method for Recording the Positions of Teeth

(71) Applicant: Przychodnia Stomatologiczna Kodent, Szczerbaniewicz Joanna, Czestochowa (PL)

(72) Inventors: Blazej SZCZERBANIEWICZ, Czestochowa (PL); Adam STACHURA, Gliwice (PL)

(21) Appl. No.: 16/413,725

(22) Filed: May 16, 2019

Related U.S. Application Data
(63) Continuation of application No. PCT/PL2018/000017, filed on Feb. 21, 2018.

(30) Foreign Application Priority Data
Feb. 22, 2017 (PL) P.420625

(51) Int. Cl. A61C 19/05; A61C 9/00; A61B 90/00

(52) U.S. Cl. CPC A61C 19/05; A61C 9/0006; A61C 9/0053; A61B 2090/395; A61B 2090/064; A61B 2090/067; A61B 90/39

(57) ABSTRACT

A measurement device for determination of fixed reference points using positioning devices (4a and 4b), para-occlusal index tray (5), changeable recording index plates/sensor (6) and impression material (15) wherein the recording process occurring via the control system (2) is initiated and stopped by means of control buttons (10) characterized in that said device consists of hoop frame (1) to be placed on the head, having a control system (2) and reference handle (3) located centrally in the frontal part and mounted at the base of the nose. A method for recording the position of teeth using the reference points initiated and stopped by means of control buttons (10) and consisting of determination of individual patient's reference points by aligning preferably the recording device (4a) or the recorded device (4b) placed on the reference handle (3) located on the hoop frame (1) placed on patient's head, to the patient's anatomy.

### CROSS REFERENCE TO RELATED APPLICATIONS

This application is a continuation under 35 U.S.C. § 120 of International Application PCT/PL2018/000017, filed Feb. 21, 2018, which claims priority to Polish Application No. P.420625, filed Feb. 22, 2017, the contents of each of which are incorporated by reference herein.

### FIELD OF THE INVENTION

The present solution relates to a measurement device and a method for recording the positions of teeth.

### BACKGROUND

A method for determination of pressure forces generated between individual teeth by means of a handle connected on one end to a computer system and on the other to changeable tips consisting of para-occlusal (peridental) index trays adjusted to the size of the dental arch and recording the pressure forces and roughly the reference positions of individual teeth; with the collected data being entered into the computer system, is known in the art.

A “T-Scan Novus” device for digital analysis of occlusion which uses and analyzes data collected in real time from such tips by means of changeable electronic index sensors connected thereto is known in the art. A device to track and visualize mandibular movements in real time that makes use of markers placed in goggles placed around the frontal part of the head and on the fork or the para-occlusal (peridental) index tray is known in the art. However, the above solution requires the use of cone beam computed tomography (CBCT) which exposes the patient to X-radiation.

A “Panadent” company device for the transfer of the locations of upper teeth relative to the temporomandibular joint is known in the art and constitutes an example element of a category of so-called facebows devices; the use of this device is possible only following manual collection and calculation of complex measurements of the patient's anatomy.

The Polish patent application no. 322920 discloses a peridental probe for the measurement of the occlusal force using changeable para-occlusal (peridental) index trays mounted in a revolving fashion on an articular joint. A method for detailed determination of the positions of individual teeth using information collected by the aforementioned devices with the additional advantage taken of computed tomography scans is known in the art. However, this solution involves the need to expose the patient to additional radiation due to the use of tomography and, due to the approximate data collected from tomographic measurements (artifacts from metal fillings within the oral cavity and facial bones), is associated with significant degree of uncertainty.

US Patent no. 2016/0030144 A discloses a method for determination of the positions of teeth using changeable index (recording) plates and a tilted inclinometer connected to that plate and mounted together with said plate on an assembly platform or, optionally, a combination of two inclinometers with one being connected to the index plate and the other being placed e.g. on patient's head so that the collected data facilitate the positioning of individual teeth not only in an analog model but also in a digital model in three planes relative to the cranium, head and/or the face following the scanning and the transfer of the model into the 3D virtual reality. The above method provided for the use of an assembly platform, with the need to manually support the index plate within the oral cavity by pressing it against the patient's teeth until complete setting of the impression material on the index plate, i.e. in a forced (fixed) position relative to the teeth themselves and to the medial line of the patient's body, thus requiring the measurement being carried out in a predefined position.

A disadvantage of all the above solutions consists in that in order to obtain accurate information on the structure and locations of individual teeth within the dental arch as well as the positions of the teeth relative to one another a number of time-consuming and inconvenient activities must be performed, e.g. by establishing a conventional point of reference since none of the aforementioned methods facilitates calculation of an individual reference point being independent from the position of the head; the solutions require additional examinations such as CT scans, and the patients undergoing the examination must adapt their positions to the index plate or the facial arch which force specific orientations of teeth and thus preclude the possibility of the examination being performed in any position of patient's head.

Another important disadvantage of the above solutions consists is that due to the lack of precisely defined reference points, the use of conventional reference points such as the infraorbital reference points is burdened by a high margin of error, making the process potentially unreliable and requiring its being repeated.

The objective of this solution is to present a measurement device and a method for recording the positions of teeth facilitating quick and precise measurement of individual patient's reference points and their mutual spatial relationship for the purposes of measuring the pressure forces and/or position of teeth and/or other variables in a single integrated process without the need of resolving to additional procedures such as CT scans and the multi-hour analysis thereof and with elimination of assembly platforms that force pre-defined positions of the head during the measurement or eliminating time-consuming and tedious manual measurements of the patient. Said measurements may be used particularly for shortening the duration of the process of quick positioning of analog/digital models in real or virtual space thanks to full dentition (i.e. upper and lower teeth) casts being positioned in an analog or virtual articulator (i.e. a device that imitates the movements of the mandible) to ensure precise spatial positioning of three-dimensional analog or virtual dentition models; additionally, the measurement device may, owing to the capability of connecting to other devices, collect information on the positions and pressure forces between the teeth upon mandibular movements e.g. by overlapping onto a space-oriented 3D model of maxillary and mandibular dentition a map of pressure forces exerted on individual teeth generated upon biting one's teeth on an electronic occlusal film with embedded sensors that record the pressure forces and transfer the measurements to a computer system.

### SUMMARY

The solution according to the present application is characterized in that said device consists of hoop frame (1) to be placed on the head, preferably in the form of an elastic band, featuring a control system (2) and reference handle (3) located centrally in the frontal part and mounted at the base of the nose, preferably equipped with recording device (4a) or recorded device (4b); and para-occlusal index tray (5) to which single-use recording index plates/sensors (6) are mounted for the recording of the shape and impression of the upper and/or lower teeth; said para-occlusal index tray preferably equipped with recorded device (4b) or recording device (4a) wherein said recording devices (4a) and recorded devices (4b) mounted to the reference handle (3) and the para-occlusal index tray (5), e.g. in the form of cameras, inclinometers, markers, distance sensors, stereoscopic cameras or other devices in various configurations should be aligned centrally along the axis of symmetry of the patient's face and are connected to the control system (2) of the device featuring control buttons (10) to initiate and complete the measurement.

The solution according to the present application is characterized in that the single-use recording index plates/sensors (6) of different sizes with fast-setting impression material (15) for the recording of both the shape and impression of upper and/or lower teeth. mounted to the para-occlusal index tray (5) may be interchangeably replaced by additional color-marking/electronic occlusal sensors recording e.g. point pressure on occlusal surface of the teeth, forces, and time of forces generated between the teeth.

The solution according to the present application is characterized in that the recording devices (4a) and the recorded device (4b) may be connected to the control system in a wired fashion or, alternatively, in a wireless fashion, e.g. by means of Bluetooth, WiFi, or other means.

The solution according to the present application is characterized in that in the first embodiment, the recording device (4a) placed on the reference handle (3) and the recorded device (4b) placed on the para-occlusal index tray (5) consist of angular position sensors, preferably in the form of inclinometers.

The solution according to the present application is characterized in that in the second embodiment, the recording device (4a) placed on the reference handle (3) consists of a camera and the recorded device (4b) placed on the para-occlusal index tray (5) consists of markers.

The solution according to the present application is characterized in that in the third embodiment, the recording device (4a) placed on the reference handle (3) consists of a camera and the recorded device (4b) placed on the para-occlusal index tray (5) consists of a marker print or sticker.

The solution according to the present application is characterized in that in the fourth embodiment, the recording device (4a) placed on the reference handle (3) consists of a device to measure distances to at least three predefined points, preferably in the form of a stereoscopic camera, allowing to determine the angular position of e.g. the reference platform identical to the actual occlusal plane, mounted to the para-occlusal index tray (5).

The solution according to the present application is characterized in that para-occlusal index tray (5) may be connected to additional devices (16) e.g. for the analysis of forces and time of forces generated between the teeth.

The solution according to the present application is characterized in that preferably the recorded devices (4b), e.g. in the form of markers placed on the para-occlusal index tray (5) may be placed directly on the additional device (16) by means of an appropriate adapter (17) for fitting different devices available on the market, thus facilitating free alignment of preferably recorded devices (4b) or recorded devices of the para-occlusal index tray (5) with the positions of preferably the recording device (4a) or a recorded device of the reference handle (3) which in turn facilitates quick and easy positioning of upper and lower teeth impressions, either real or virtual, on the basis of the measurements taking into consideration the parameters obtained using the additional devices (16).

The solution according to the present application is characterized in that preferably the recorded device (4b) or the recording device (4a) placed on the para-occlusal index tray (5) features a universal socket (4c) for fitting para-occlusal index trays of different sizes available on the market.

The solution according to the present application is characterized in that adapter (17) in the form of a surface preferably with a mounted recorded device (4b) features matches the shape of the housing of the additional device (16) wherein said surface features offsets in the form of at least two jackets for the placement of the additional device (16) ensuring stable connection of preferably the recorded device (4b) and the additional device (16).

The solution according to the present application is characterized in that the control system (2) is directly connected in a wired or wireless fashion to an external device, e.g. a computer or a telephone containing the necessary algorithm for generation of a virtual model of upper jaw and for positioning said model in a real-life or virtual articulator.

The solution according to the present application is characterized in that the reference handle (3) preferably containing a hygienic rubber pad at the point of contact between the handle and the patient's head features a reference index plate (3¹) preferably with linear graduation scale, for easier alignment of the recording device placed on the reference handle for possibly best measurement of the reference point, an articulated joint, preferably a ball-and-socket joint (3²) which, when used in combination with the linear graduation, facilitates positioning of the preferably recording device (4b) so as to possibly most precisely determine the individual reference points for the vertical and the horizontal axis of patient's head, and the positioning handle (3³) mounted at the base of the nose.

The solution according to the present application is characterized in that the reference handle (3) features two openings (3⁴) to facilitate the mounting of the reference handle (3) to the hoop frame (1) by means of threading the hoop through said openings.

The solution according to the present application is characterized in that the preferably the recording device (4a) placed on the reference handle (3) is enclosed in a housing (9) featuring a socket (9¹) for the placement of the recording device (4a), an articulation joint socket (9²) for the placement of housing (9) on the reference handle (3) and preferably an indicator (9³) to facilitate the positioning of the device with the help of the linear graduation scale on the reference handle (3).

The solution according to the present application is characterized in that at least two positioning peripherals (14) are connected to the para-occlusal index tray (5) at both of its branches for better allocation of the para-occlusal index tray (5), said peripherals being covered by impression material (15) so that following the placement of the para-occlusal index tray (5) with the positioning peripherals (14) covered with the impression material (15) into the oral cavity and following the setting of the impression material, an unambiguous positioning of upper teeth may be obtained in partly or full edentulous patients.

The solution according to the present application is characterized in that the positioning peripheral (14) consists of at least one rib in the form of two columns connected by a curved crosspiece located at about one half of the columns' height, the width of the crosspiece being greater than the width of the column to facilitate the placement of the positioning peripheral (14) on the para-occlusal index tray (5).

The solution is characterized in that the method for recording the position of teeth consists of determination of individual patient's reference points by matching preferably the recording device (4a) or the recorded device (4b) placed on the reference handle (3) located on the hoop frame (1) placed on patient's head to the patient's anatomy, preferably using linear graduation scale placed on the reference handle and the indicator (9³) on housing (9) and aligning preferably the recording device (4a) or the recorded device (4b) to the axis of symmetry of the patient's face, followed by pairing preferably the recording device (4a) or the recorded device (4b) located on the reference handle (9³) with preferably the recorded device (4b) or recording device (4a) located outside the patient's mouth on the para-occlusal index tray (5) which in its part located inside the patient's mouth is equipped with a changeable recording index plate/sensor (6) covered with impression material (15) to be bitten by the patient so that the impression material, when hardened, remembers the shape and impression of upper and/or lower teeth and ensures stable positioning of the para-occlusal index tray (5) so that after hardening of the impression material both devices (4a and 4b), i.e. the devices located on the reference handle (3) and the para-occlusal index tray (5) are centrally aligned to the axis of symmetry of the patient's face in a defined anatomical relationship being recorded by the recording system (2) so that both devices (4a and 4b) define unambiguous, fixed reference points determined by angular measurement of the position of upper teeth in space as well as in relation to the head, i.e. a mutual, unambiguously and simultaneously defined relationship between those by taking into consideration angular measurements consisting of determination of differences in the angular position of para-occlusal index tray (5), preferably connected to a recording index plate (6) and the reference point, with the data originating from both devices (4a and 4b), i.e. the devices located on the reference handle (3) and the para-occlusal index tray (5) are transmitted to the control system (2) permitting their use for determination of the geometrical position of the upper jaw of the patient unambiguously to the position of the patient's head.

The solution is characterized in that the method for recording the position of teeth making use of the additional device (6), such as a scanner or another device, consists of mounting said additional device (16) to the para-occlusal index tray (5) followed by preferably the recorded device (4b) being allocated directly on the additional device (16) so that both devices (4a and 4b), i.e. the devices located on the reference handle (3) and the additional device (16) connected to the para-occlusal index tray (5) preferably featuring an (electronic) occlusal sensor, are aligned to the axis of symmetry of the patient's face in a fixed anatomical relationship to each other so that the collected information would be supplemented by information from the additional device (16) such as information on point pressure on the occlusal surface of the teeth, forces, and time of forces generated between the teeth and, together with information collected by the recording device (4a) and the recorded device (4b), sent to the control system (2) thus facilitating their use not only in the positioning of the upper dentition model in the form of mounted model or a digital model but also, in the case of the digital model, unambiguous assignment to the occlusal surfaces of the upper and/or lower teeth real points that provide precise spatial reflection of the pressure forces generated upon biting one's teeth or moving one's lower jaw along with the temporal sequence of generation of these forces.

An advantage of the solution according to the present application consists in the ability of quick and precise definition of fixed individual reference points by means of determination of the spatial relationship of the head and the upper teeth in an individual patient without the need to perform additional procedures such as computed tomography; in that the measurement is performed in a natural position of the head and with patient's natural behavior of the lower jaw (mandible), thus markedly shortening the measurement process, eliminating the margin of error caused by the imprecise, conventional reference points; and finally in the greater convenience of the measurement as the troublesome positioning of the patient's head using the indexing (recording) plates and the need to perform lengthy measurements is eliminated, and in lower negative impact on patient's health due to the avoided exposure to radiation in tomographic scans.

Another advantage of the solution according to the present application is that the solution provides for the possibility to connect additional external devices (16) to facilitate generation of additional information, e.g. an the pressure forces being generated on the occlusal surfaces of individual teeth upon biting, the temporal sequence of generation of these forces as well as individual movements and chewing movements, in a single integrated process.

Another advantage of the solution according to the present application is the possibility to use the data obtained from the device in real time during the measurement to determine the mutual relationship of the positions of the upper and lower jaw to be transferred as mounted models to an analog articulator or as digital models to a virtual articulator.

Another advantage of the solution according to the present application is the ability of making use of additional information collected upon clinical examination of the patient using the bite registration index plate which, when covered with any bite registration material may, after setting, be used as a bite registration key for the alignment of plaster (mounted) models (casts) of the maxilla and the mandible in an analog articulator or, after subsequent scanning, as a virtual bite registration key for the alignment of digital models of the maxilla and the mandible in a virtual articulator.

### BRIEF DESCRIPTION OF SEVERAL VIEWS OF DRAWINGS

The subject of the solution is presented as an example embodiment and illustrated in figures FIGS. 1-13.

FIG. 1 shows an example embodiment of the measurement device taking advantage of the positioning devices, i.e. the recording device (4a) and the recorded device (4b) consisting of an assembly of two inclinometers connected by cables (8) to the control system (2) and placed on the hoop frame (1) with the reference handle (3) and para-occlusal index tray (5) placed on the upper jaw (7) wherein the analysis/measurement is initiated and completed by means of control buttons (10).

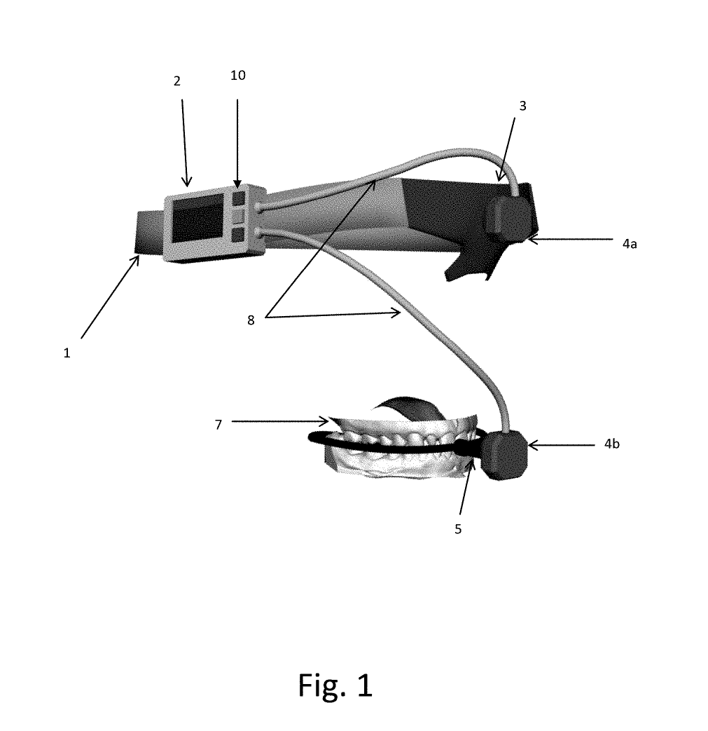

FIG. 2 shows an example embodiment of the reference handle (3) with the reference plate (3¹) equipped with a ball-and-socket joint (3²) for the placement of the recording device (4a) and openings (3⁴) for mounting the reference handle (3) to the hoop frame (1), said reference handle featuring in its bottom part (3) a handle (3³) for the placement of the reference handle (3) on patient's nose.

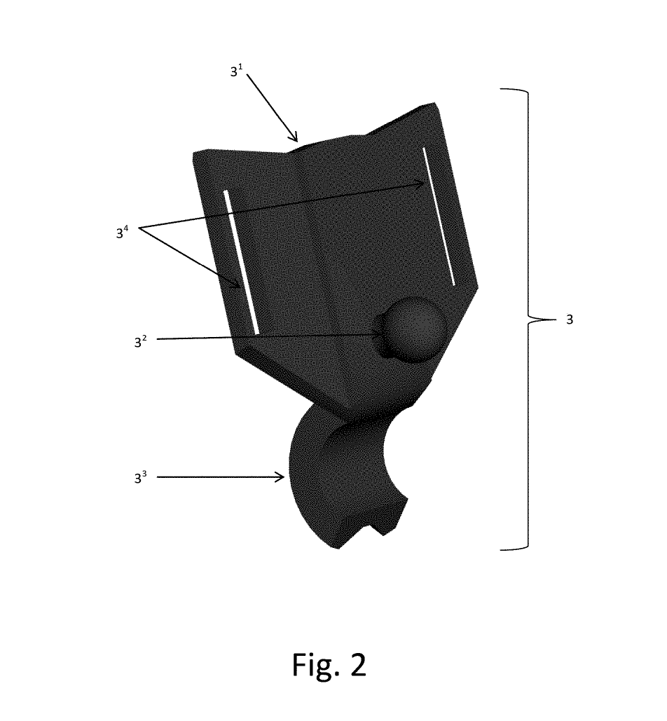

FIG. 3 shows an example embodiment of the housing (9) in side view, featuring a socket (9¹) for the placement of the recording device (4a), an articulation joint socket (9²) for the placement of housing (9) on the reference handle (3) and preferably an indicator (9³) to facilitate the alignment of the device with the individual axis of symmetry of the patient's face with the help of the linear graduation scale on the reference handle (3).

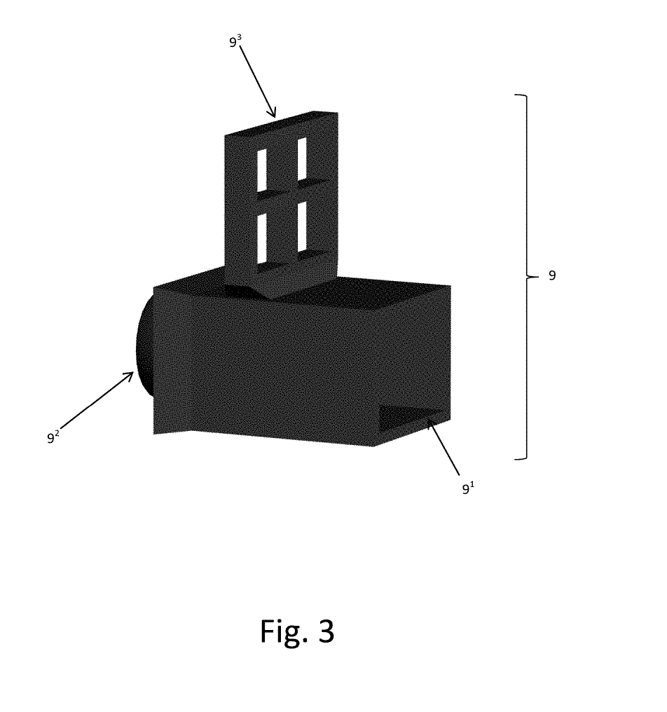

FIG. 4 shows an example embodiment of the housing (9) in back view with a close-up of the articulation joint socket (9²) for the mounting of the housing (9) to the reference handle (3), and of the indicator (9³) equipped with a socket (9¹) for the placement of the recording device (4a) which constitute the base of the housing (9).

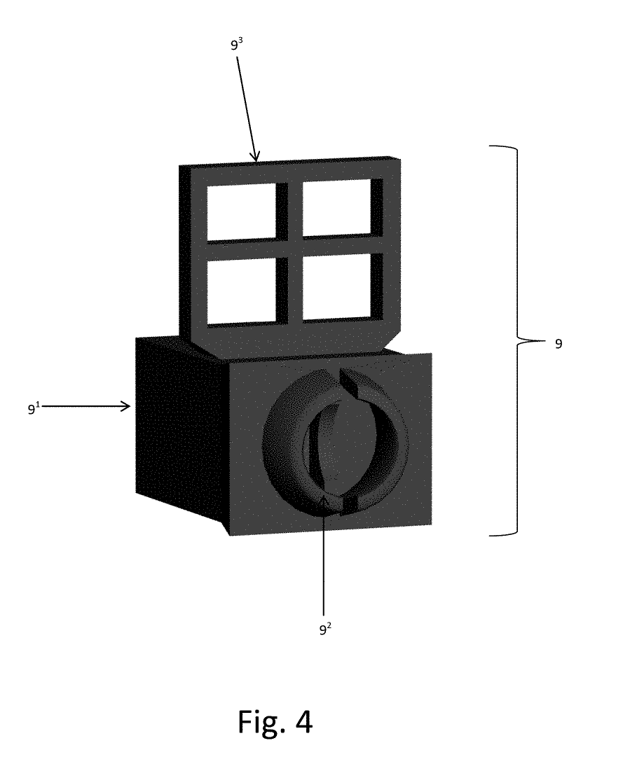

FIG. 5 shows an example embodiment of the reference handle (3) with housing (9) mounted thereto.

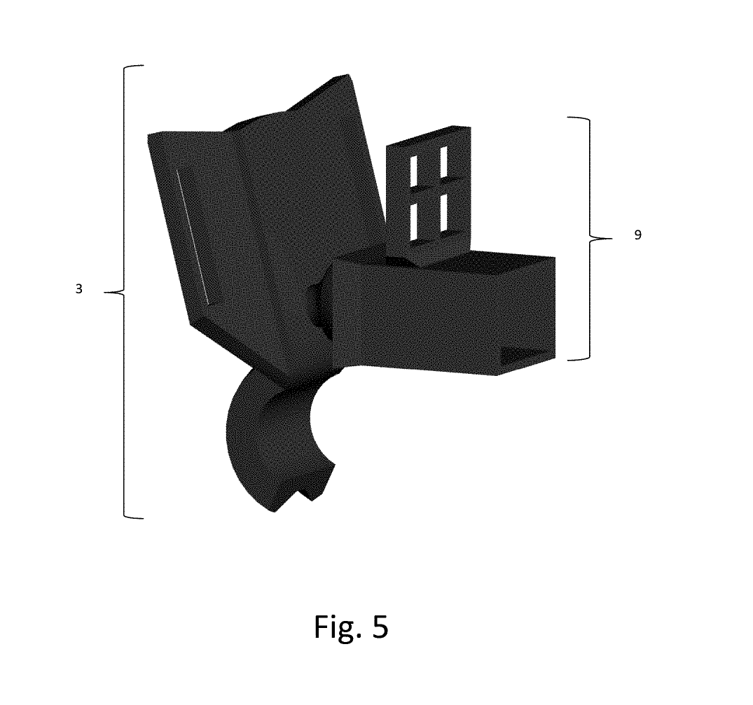

FIG. 6 shows an example embodiment of the para-occlusal index tray (5) with the recording index plate/sensor (6) and the recorded device (4b) featuring an universal socket (4c) which, after being covered with a fast-setting impression material for the recording of the shape and impression of the upper and lower teeth of the patient is placed in patient's mouth until the hardening of the fast-setting impression material and subsequently removed and transferred to an analog or virtual articulator.

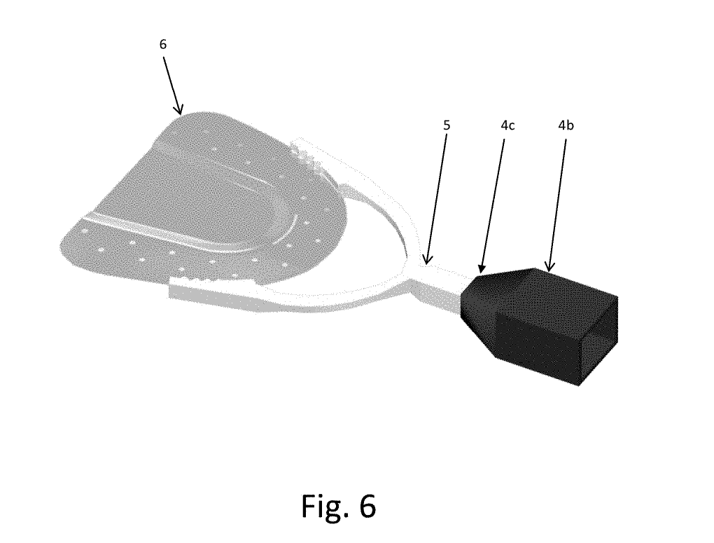

FIG. 7 shows a model of the patient (11) with the device adjusted to fit his/her anatomy and consisting of the elements detailed above such as the hoop frame (1), the recording device (4a) within a housing (9) mounted to the reference handle (3) and the recorded device (4b) on the para-occlusal index tray (5), said recording devices being connected by cables (8) to the control system (2) featuring control buttons (10) (to initiate and stop the measurement).

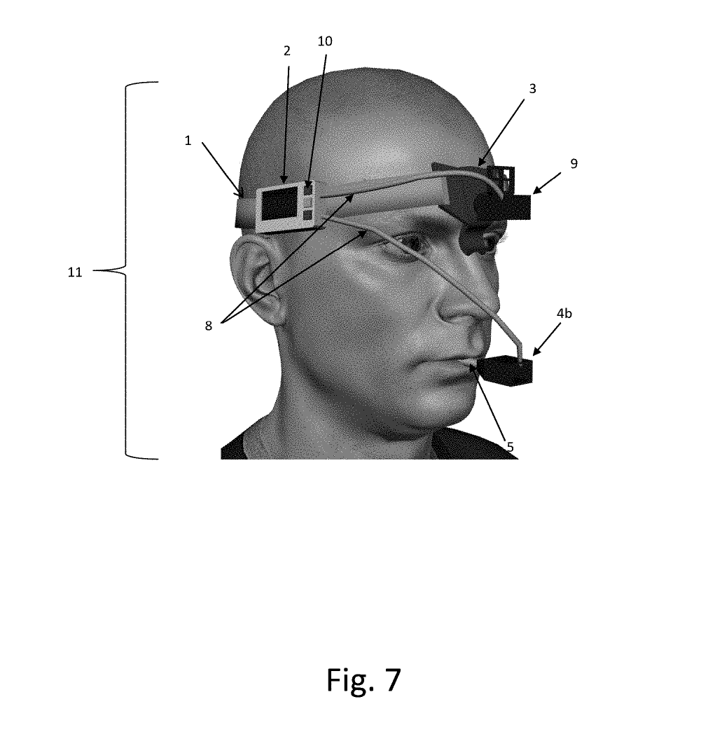

FIG. 8 shows a model of the patient (11) as well as visualization of the planes (12) and (13) of the reference measurement (reference planes) made by the device and used to map the anatomy of the patient for use in further processes such as obtaining information on the positions of the teeth, the temporal sequence of contact between teeth, the pressure forces generated between the teeth, etc., by means of data processes methods known in the art.

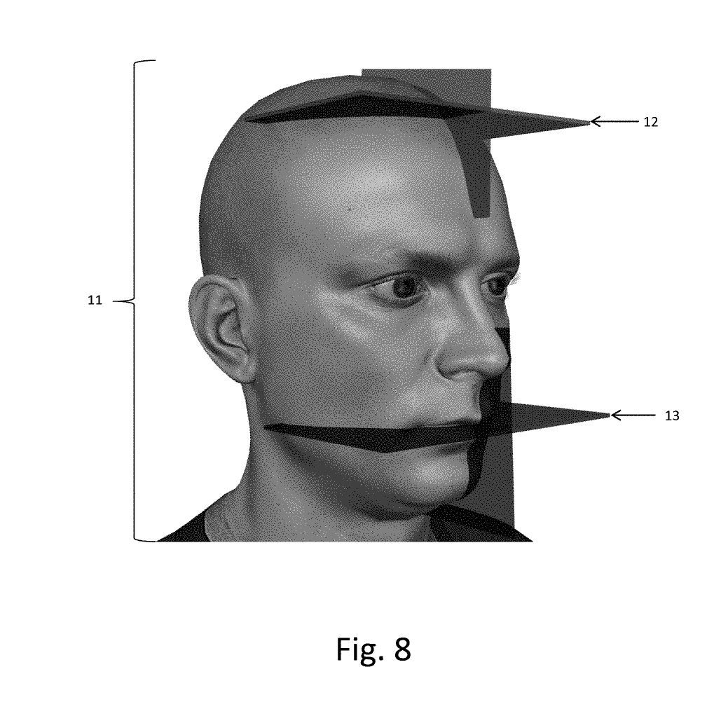

FIG. 9 shows an example embodiment of the procedure of preparation of the para-occlusal tray (5) before placing the tray within the patient's mouth, with identification of two positioning peripherals (14), their connection to the para-occlusal index tray (5), and their sealing using the impression material (15) wherein the presented embodiment features a positioning peripheral (14) consisting of a double rib, i.e. three poles connected by two crosspieces.

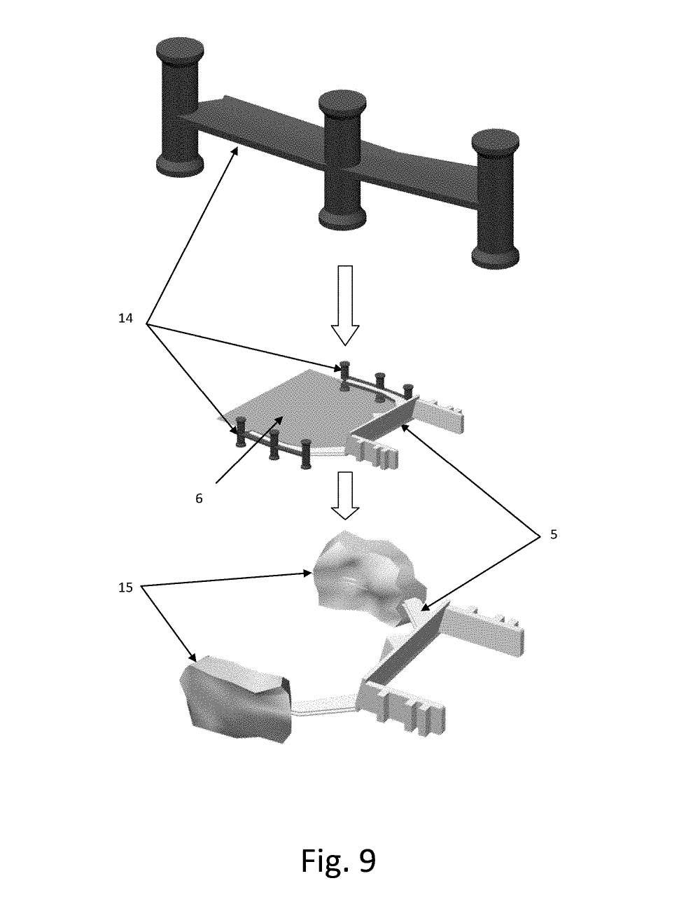

FIG. 10 shows a transparent view of an example embodiment of the patient model (11) with the device adjusted to fit his/her anatomy and consisting of the elements detailed above such as the hoop frame (1) with the control system (2) featuring control buttons (10), the recording device (4a) consisting of a camera mounted to the reference handle (3) connected by cables (8) to the control system (2), and the para-occlusal index tray (5) with the recording index plate/sensor (6) which may additionally be furnished with e.g. an electronic sensor to record the pressure forces generated between the teeth of the upper and the lower dental arch and the temporal sequence of generation thereof, with identification of the positioning peripherals (14), their connection to the para-occlusal tray branches, and the additional device (16) to which the recorded device (4b) consisting of stuck on marker is mounted wherein, thanks to the transparent view of the patient model, the para-occlusal index tray (5) and the positioning peripherals (14) are visualized as placed within the patient's mouth.

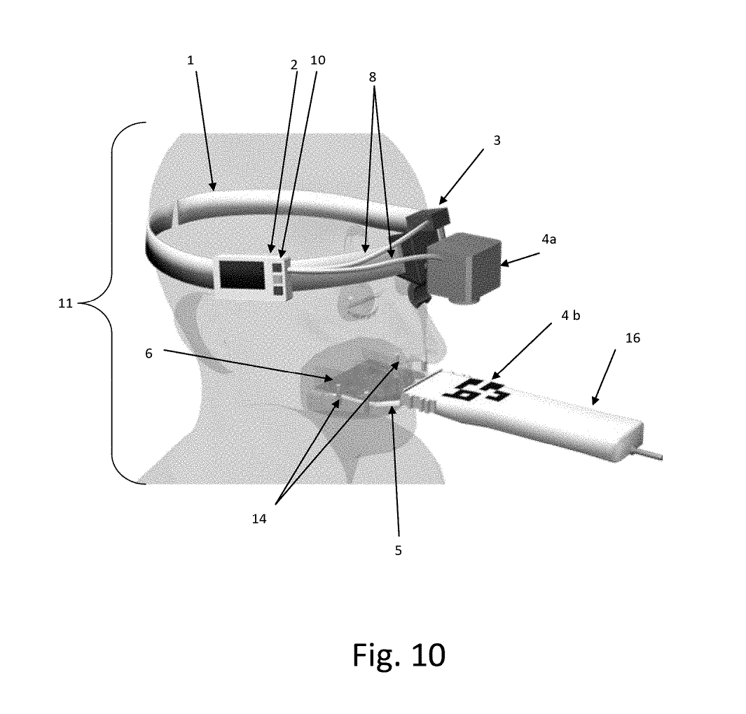

FIG. 11 shows an example embodiment of the procedure to be followed during allocation of the recorded device (4b) consisting of markers on the additional device (16) using the adapter (17).

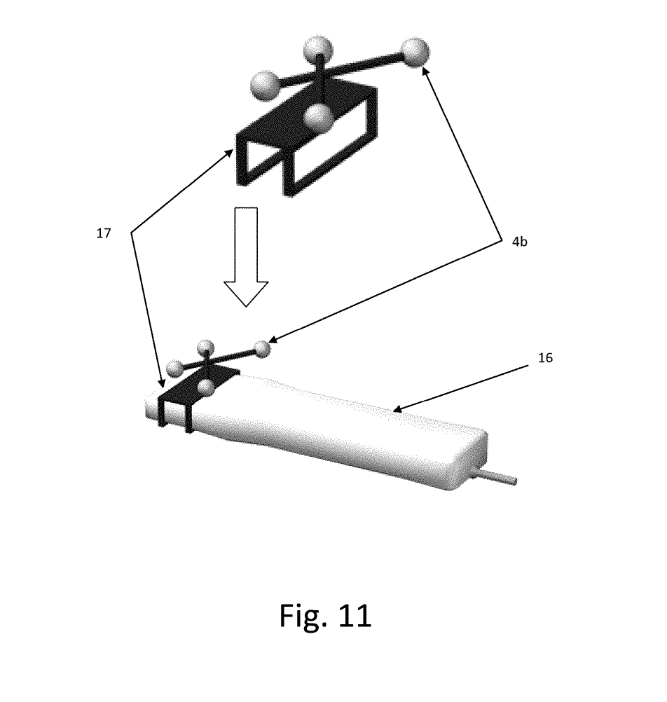

FIG. 12 shows a transparent view of an example embodiment of the patient model (11) with the device adjusted to fit his/her anatomy and consisting of the elements detailed above such as the hoop frame (1) along with the recording device (4a) consisting of a camera connected by cables (8) to the control system (2) with control buttons (10), mounted to the reference handle (3) and the para-occlusal index tray (5) with particular identification of the impression material (15) placed on the branches of the para-occlusal index tray and the additional device (16) to which the recorded device (4b) consisting of markers are mounted using an adapter wherein, thanks to the transparent view of the patient model, the para-occlusal index tray (5), the recording index plate/sensor (6) which may additionally be furnished with e.g. an electronic sensor to record the pressure forces generated between the teeth of the upper and the lower dental arch, and the impression material (15).

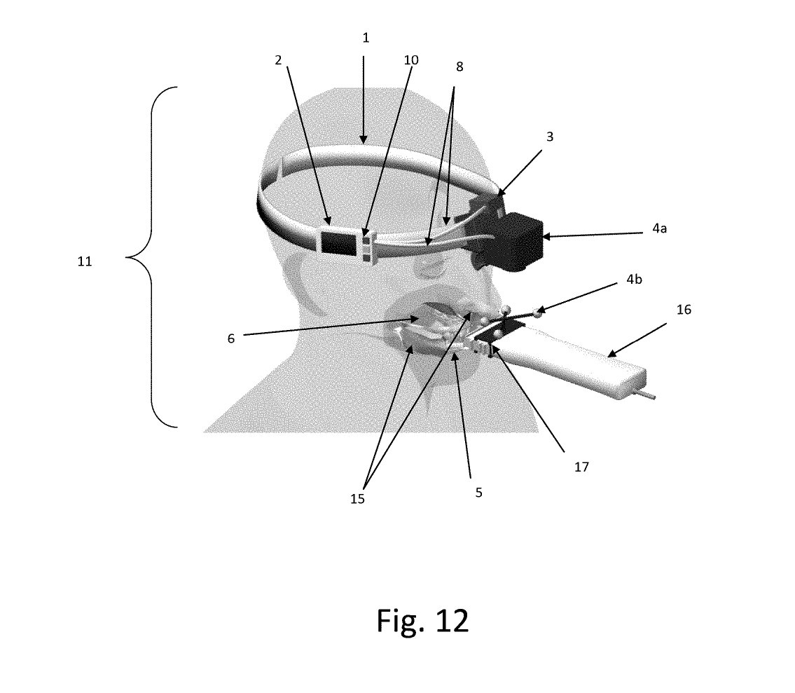

FIG. 13 shows an example embodiment of an individual model of the upper and lower jaw (7) generated using the recording device (4a) and the recorded device (4b) in a digital articulator wherein a map of the impression and mutual spatial relationships between the teeth were collected upon scanning of the bite recording plate/sensor (6) and the hardened impression material (15) in a single integrated measurement using the device according to the solution as shown in the figure. The obtained positions may be used for subsequent restoration or completion of missing dental structures or for mapping (restore) the chewing patterns.

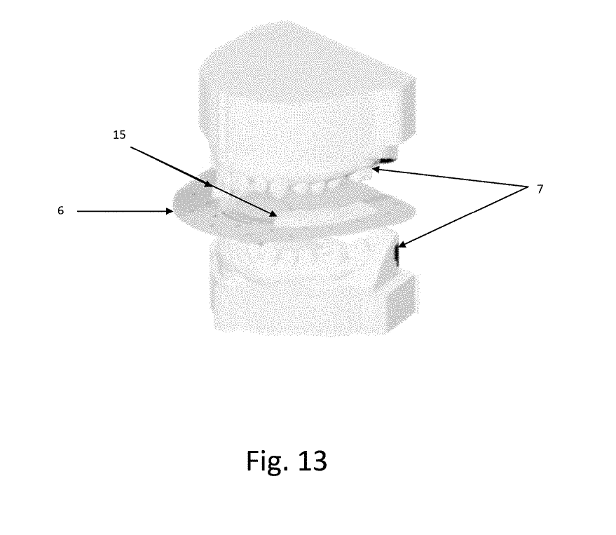

What is claimed is:

1. A measurement device for determination of fixed reference points using
positioning devices (4a and 4b);
a para-occlusal index tray (5);
a changeable recording index plates/sensor (6); and
an impression material (15), wherein
the recording process occurring via the control system (2) is initiated and stopped by means of control buttons (10), characterized in that said device consists of hoop frame (1) to be placed on the head, in the form of an elastic band consisting a control system (2) and
reference handle (3) located centrally in the frontal part and mounted at the base of the nose, equipped with recording device (4a) or recorded device (4b), and para-occlusal index tray (5) to which single-use recording index plates/sensors (6) are mounted and on which the impression material (15) is placed, wherein the para-occlusal index tray equipped with recorded device (4b) or recording device (4a), wherein said recording devices (4a) and recorded devices (4b), mounted to the reference handle (3), and the para-occlusal index tray (5) during the measurement should be aligned centrally along the axis of symmetry of the patient's face, and are connected to the control system (2).

2. The measurement device according to claim 1, wherein the single-use recording index plates (6) of different dimensions with fast-setting impression material (15) for the recording of both the shape and impression of upper and/or lower teeth, mounted to the para-occlusal index tray (5), may be interchangeably replaced by additional color-marking/electronic occlusal sensors recording e.g. point pressure on occlusal surface of the teeth, forces, and time of forces generated between the teeth.

3. The measurement device according to claim 1, wherein the recording devices (4a) and the recorded device (4b) may be connected to the control system in a wired fashion or, alternatively, in a wireless fashion, e.g. by means of Bluetooth, WiFi, or other means.

4. The measurement device according to claim 1, wherein the recording device (4a) placed on the reference handle (3) and the recorded device (4b) placed on the para-occlusal index tray (5) consist of angular position sensors in the form of inclinometers.

5. The measurement device according to claim 1, wherein the recording device (4a) placed on the reference handle (3) consists of a camera and the recorded device (4b) placed on the para-occlusal index tray (5) consists of markers.

6. The measurement device according to claim 1, wherein the recording device (4a) placed on the reference handle (3) consists of a camera and the recorded device (4b) placed on the para-occlusal index tray (5) consists of a marker print or sticker.

7. The measurement device according to claim 1, wherein the recording device (4a) placed on the reference handle (3) consists of a device to measure distances to at least three predefined points in the form of a stereoscopic camera.

8. The measurement device according to claim 1, wherein the para-occlusal index tray (5) is connected to an additional device (16).

9. The measurement device according to claim 1, wherein the recorded device (4b) placed on the para-occlusal index tray (5) is placed directly on the additional device (16) using an appropriate adapter (17).

10. The measurement device according to claim 1, wherein the recorded device (4b) or the recording device (4a) placed on the para-occlusal index tray (5) features a universal socket (4c) for fitting para-occlusal index trays of different dimensions available on the market.

11. The measurement device according to claim 1, wherein the adapter (17) in the form of a surface with a mounted recorded device (4b) has offsets in the form of at least two jackets for the placement of the additional device (16).

12. The measurement device according to claim 1, wherein the control system (2) is directly connected in a wired or wireless fashion to an external device containing the necessary algorithm.

13. The measurement device according to claim 1, wherein the reference handle (3) containing a hygienic rubber pad at the point of contact between the handle and the patient's head features a reference index plate (3¹) with linear graduation, an articulated joint, preferably a ball-and-socket joint (3²), and the handle(3³).

14. The measurement device according to claim 1, wherein the reference handle features two openings (3⁴).

15. The measurement device according to claim 1, wherein the recording device (4a) placed on the reference handle (3) is enclosed in a housing (9) featuring a socket (9¹), an articulation joint socket (9²) and an indicator (9³).

16. The measurement device according to claim 1, wherein at least two positioning peripherals (14) are connected to the para-occlusal index tray (5) at both of its branches by means of the impression material (15) so that following the placement of the para-occlusal index tray (5) with the positioning peripherals (14) covered with the impression material (15) into the oral cavity and following the setting of the impression material, an unambiguous positioning of upper teeth may be obtained.

17. The measurement device according to claim 1, wherein the positioning peripheral (14) consists of at least one rib in the form of two columns connected by a curved crosspiece located at about one half of the columns' height, the width of the crosspiece being greater than the width of the column.

18. A method for recording the position of teeth using the reference points initiated and stopped by means of control buttons (10), wherein said process consists of:
determination of individual patient's reference points by aligning the recording device (4a) or the recorded device (4b), placed on the reference handle (3) located on the hoop frame (1) placed on patient's head, to patient's anatomy, using linear graduation placed on the reference handle and the indicator (9³) on housing (9) and aligning the recording device (4a) or the recorded device (4b) to the axis of symmetry of the patient's face;
pairing the recording device (4a) or the recorded device (4b) located on the reference handle (3) with the recorded device (4b) or recording device (4a) located outside the patient's mouth on the para-occlusal index tray (5) which in its part located inside the patient's mouth is equipped with a changeable recording index plate/sensor (6) covered with impression material (15);
biting of the impression material (15) by the patient;
remembering the shape and impression of upper and/or lower teeth after the impression material is set;
ensuring stable positioning of the para-occlusal index tray (5) so that after the setting of the impression material both devices (4a and 4b), i.e. the devices located on the reference handle (3) and the para-occlusal index tray (5) are centrally aligned to the axis of symmetry of the patient's face in a defined anatomical relationship;
recording the anatomical relationship by the recording system (2) so that both devices (4a and 4b) define unambiguous, fixed reference points determined by angular measurement of the position of upper teeth in space as well as in relation to the head, i.e. a mutual, unambiguously and simultaneously defined relationship between those;
taking into consideration angular measurements consisting of determination of differences in the angular position of para-occlusal index tray (5) connected to a recording index plate (6) and the reference point; and
transmitting data originating from both devices (4a and 4b), i.e. the devices located on the reference handle (3) and the para-occlusal index tray (5) to the control system (2) permitting their use for determination of geometrical relationship between the positions of the upper jaw of the patient unambiguously to the position of the patient's head.

19. The method for recording the position of teeth according to claim 18, wherein when an additional device (16) is used said process consists additionally of:
mounting the said additional device (16) to the para-occlusal index tray (5); and
allocation of the recorded device (4b) directly on the additional device (16) so that both devices (4a and 4b), i.e. the devices located on the reference handle (3) and the additional device (16) connected to the para-occlusal index tray (5), featuring an electronic occlusal sensor, are aligned to the axis of symmetry of the patient's face in a fixed anatomical relationship to each other.
